(19) United States
(12) Patent Application Publication
(10) Pub. No.: US 2020/0375527 A1
(43) Pub. Date: Dec. 3, 2020

(54) METHOD AND SYSTEM FOR OPTIMIZING SHORT-TERM SLEEP

(71) Applicant: FUTURE WORLD HOLDINGS LLC, Burlingame, CA (US)

(72) Inventors: Raghu Bathina, Los Altos Hills, CA (US); Sridhar Prathikanti, Burlingame, CA (US)

(21) Appl. No.: 16/425,946

(22) Filed: May 30, 2019

(51) Int. Cl. A61B 5/00; G06N 3/08; G06N 7/00; G06N 20/20; G06F 3/01; G02B 27/01

(52) U.S. Cl. CPC A61B 5/4815; A61B 5/7435; G06N 3/08; G06F 2203/011; G06N 20/20; G06F 3/011; G02B 27/017; G06N 7/005

(57) ABSTRACT

A method for facilitating a sleep session is provided. The method comprises provisioning a virtual reality device with at least one sleep program configured to provide an immersive experience to facilitate a sleep session for a user; determining recommended sleep settings for the sleep program; configuring the sleep program with at least some of the recommended sleep settings; running the sleep program; and tracking sleep information for the user associated with the sleep session.

### FIELD

Embodiments of the present invention relate to sleep.

### BACKGROUND

There have been efforts to use technology in order to improve sleep quality. For example, mobile phones, wearable devices such as the Apple Watch, and the Fitbit have the capability to measure various aspects of sleep such as sleep duration, sleep quality, etc.

### SUMMARY

According to a first aspect of the invention, there is provided a method for facilitating a sleep session, the method comprising: provisioning a virtual reality device with at least one sleep program configured to provide an immersive experience to facilitate a sleep session for a user; determining recommended sleep settings for the sleep program; configuring the sleep program with at least some of the recommended sleep settings; running the sleep program; and tracking sleep information for the user associated with the sleep session.

According to a second aspect of the invention, there is provided a system for facilitating a sleep session, the system comprising: a virtual reality device provisioned with at least one sleep program configured to provide an immersive experience to facilitate a sleep session for a user; a server device communicatively coupled to the virtual reality device and configured to provide recommended sleep settings for the sleep program.

Other aspects of the invention will be apparent from the written description below.

### BRIEF DESCRIPTION OF THE DRAWINGS

FIG. 1 is a schematic drawing illustrating an implementation of a sleep system, in accordance with one embodiment of the invention.

FIG. 2 is a schematic drawing illustrating a client application for generating sleep recommendations and facilitating a short-term sleep session, in accordance with one embodiment of the invention.

FIG. 3 is a schematic drawing illustrating a server application for generating sleep recommendations in accordance with one embodiment of the invention.

FIG. 4 shows exemplary sleep settings for a sleep session, in accordance with one embodiment of the invention.

FIGS. 5A and 5B show a flow chart of operations in accordance with one embodiment of the invention, to generate recommended settings for a sleep session.

FIG. 6 shows a high-level block diagram of hardware for implementing a server device of the inventive sleep system, in accordance with one embodiment of the invention.

### DETAILED DESCRIPTION

In the following description, for purposes of explanation, numerous specific details are set forth in order to provide a thorough understanding of the invention.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not others.

Moreover, although the following description contains many specifics for the purposes of illustration, anyone skilled in the art will appreciate that many variations and/or alterations to said details are within the scope of the present invention. Similarly, although many of the features of the present invention are described in terms of each other, or in conjunction with each other, one skilled in the art will appreciate that many of these features can be provided independently of other features. Accordingly, this description of the invention is set forth without any loss of generality to, and without imposing limitations upon, the invention.

As will be appreciated by one skilled in the art, the aspects of the present invention may be embodied as a system, method or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “module,” or “system.” Furthermore, aspects of the present invention may take the form of a computer program product embodied in one or more computer readable medium(s) having computer readable program code embodied thereon.

Embodiment of the present invention disclose a system to improve the quality of a person's sleep, particularly naps or short-term sleep.

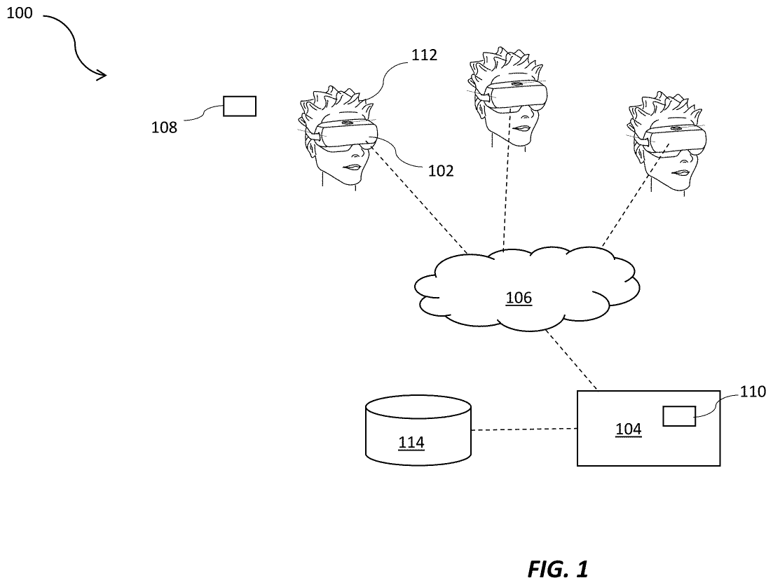

An implementation 100 of the inventive system is shown in FIG. 1 of the drawings. Referring to FIG. 1, it will be seen that the system 100 includes a virtual reality device 102 For example, the virtual reality device may be the device sold under the tradename Oculus Go. The virtual reality device 102 is provisioned with client software 108 configured to provide recommendations to a user in order to improve said user's short-term sleep, as will be described. In a typical deployment, there may be several virtual reality devices 102, each worn by a user 112.

As will be seen, the virtual reality devices 102 are communicatively coupled to a server device 104 by means of an intermediate network, such as a wide area network 106. The server device 104 is provisioned with server software 110 configured to implement functions to improve a user's sleep in collaboration with the client software 108, as will be explained. The network 106, which in some embodiments may comprise the Internet, facilitates data exchange between the client software 108 and the server software 110.

In use, when a user 112 wants to take a nap, said user puts on the virtual reality device 102, as shown in FIG. 1 and launches the client software 108. Functionally, the client software 108 provides programs that include a plurality of virtual reality environments that are designed to induce, support, or otherwise facilitate sleep. Each of the programs may be customized by a user through a plurality of settings. These settings may be selected by a user or may be provided in the form of a recommendation from the server app/software 110. In one embodiment, the settings may include choices for background music, sleep duration, visual scenes, etc.

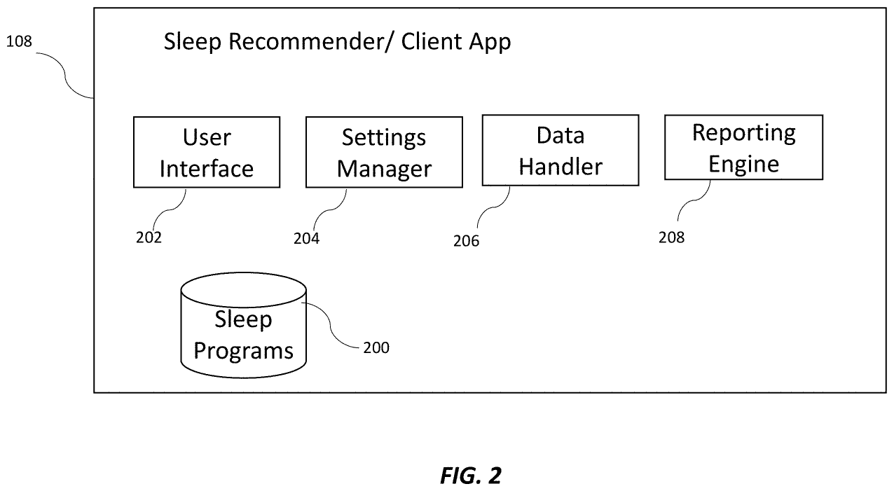

FIG. 2 of the drawings shows a high-level functional block diagram of components of the client software 108, in accordance with one embodiment. As will be seen, the client software 108 includes a database 200 configured to store a plurality of sleep programs. Each sleep program is designed to provide an immersive experience to help a user to take a nap. According to various embodiments, each sleep program may include choices/settings to control a visual environment, environmental sound volume, background music, background music volume, theta waves (controls may be provided to turn theta waves on or off according to user preferences) and a desired nap time (for example 20 minutes). The visual environment comprises the images that a user would see. For example, the environment could include a beach scene, or a campfire scene. Environmental sounds may include the particular sounds associated with scenes from the visual environment. For example, if the scene includes a campfire, then the environment found may include a crackling sound of the type typically made by a campfire.

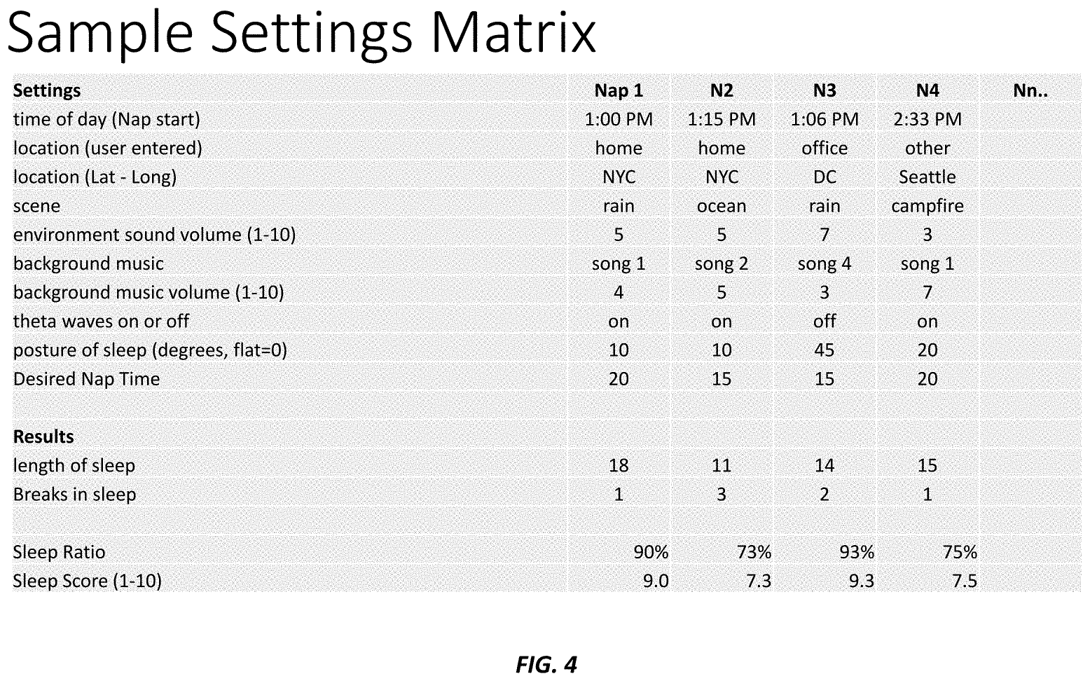

The client software 108 further comprises a user interface component 202, a settings manager 204, a data handler 206, and a reporting engine 208. In one embodiment, the user interface may include control to enable the user to select settings for a particular sleep session. Referring now to FIG. 4 of the drawings, there is shown a sample sleep settings matrix, in accordance with one embodiment of the invention. As will be seen, the sleep settings include the following settings: time of day (nap start time); location (user entered); location (in terms of latitude and longitude), scene, environment sound volume (1-10), background music volume (1-10), theta waves on or off, posture of sleep (expressed in terms of an inclination in degrees, where a reflect sleep posture is assigned 0°), and desired nap time.

In some embodiments, some sleep settings may be obtained from system components, and settings. For example, the time of day setting may be obtained from a system clock, the location setting in terms of latitude and longitude may be obtained from a location sensor, and the posture of sleep may be inferred from information from a gyroscope.

In one embodiment, all settings for a particular sleep session may be managed by the settings manager 204. As noted above, the settings manager 204 may be configured to obtain at least some of the sleep settings from the user via the user interface component 202. The settings manager 204 may also be configured to obtain sleep settings for a particular sleep session from the server software 110, as will be explained later.

In one embodiment, a gyroscope in the virtual reality device 102 measures a user's head movement in three axes, and this data may be used to determine when a user falls asleep and when the user wakes up.

The data handler component 206 is responsible for data exchanges between the client software 108, and the server software 110. Typically, data exchange between these two components include sleep session settings, and sleep/nap session data.

After a nap session is completed, session data for the nap is transmitted via the reporting engine 208 to the server software 110 via the data handler 206 so that a sleep profile maintained for the user by the client/server software 110 may be updated.

In one embodiment, the data for a particular sleep session may be shared with the user by means of the user interface 202. In particular, the user may be able to view and act on statistics and recommendations for particular sleep settings received from the server software 110, as will be explained. Thus, for example, for a future sleep session, a user can choose the recommended sleep settings instead of manually configuring these settings.

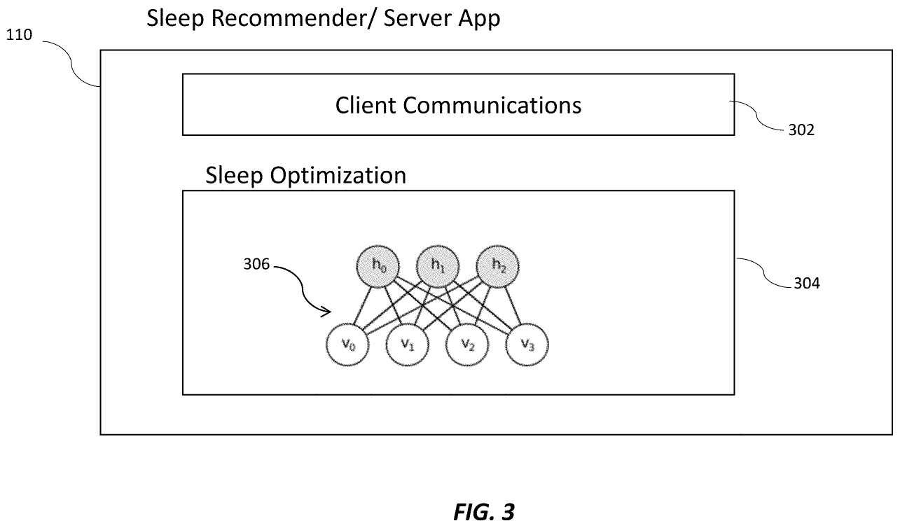

FIG. 3 of the drawings shows a high-level functional block diagram of components for the server software 110 in accordance with one embodiment of the invention. As will be seen, the server software 110 includes a client communications module 302. The purpose of this module is to maintain communications with the client software 108 of each virtual reality device 102 that is part of the system. Thus, part of the function of the client communications module 302, in one embodiment, is to facilitate a data exchange with each virtual reality device 102. Said data exchanges may include sleep data, and sleep session setting recommendations.

The server software 110, also includes a sleep optimization component 304 configured to analyze sleep session data for each user and to generate sleep session recommended settings for said user based on the analysis. In one embodiment, the sleep optimization component 304 may implement machine learning algorithms in order to derive the ideal conditions for “good” sleep given a user's history of sleeping patterns. In one embodiment, said learning algorithms may be implemented as a neural network. For this purpose, x1, x2, . . . xN may define a set of observable features affecting a person's sleep, which can be described a vector X of length “n”. In this case, a quality of sleep Y for each combination of x's over the person's sleeping history may be computed. In formal terms, the problem of determining optimal sleep session settings may be resolved into the goal of maximizing P(X|Y=y), where y equals the desired quality of sleep. In one embodiment, the set of observable features corresponds to the sleep session settings associated with the sleep programs.

Maximizing P(X|Y=y) may be achieved by implementing based on Energy Based Models (EBM), and Restricted Boltzman Machines (RBM).

Energy Based Models (EBM) are a class of Energy based models, configured to associate a scalar energy to each configuration of the variables. Learning corresponds to modifying that energy function so that its shape has desirable properties. The desired or plausible configurations should have low energy levels. Energy based probabilistic models define a probability function through an energy function, as follows:

P(x)=e−E(x)/Z

Where the normalizing factor Z is called the partitioning function as described in physical systems.

Z=Σe−E(x)

Restricted Boltzmann Machines (RBM) are a particular form of log linear Markov Random Field (MRF) i.e. for which the energy function is linear in its free parameters.

In FIG. 3, the sleep optimization component 304 is shown to include a Restricted Boltzman Machine 306 for purposes of determining the optimal sleep session parameters for each user. Typically, the computation for determining the optimal sleep session parameters includes analysis of data collected across multiple past sleep sessions. In one embodiment, a result of said analysis may include, but is not limited to:

general statistics: longest duration, total sleep minutes this week, best sleep day, etc.

recommended settings: best time of the day for sleep, best visual for sleep, best music and volume settings for optimal sleep. These settings achieve the highest sleep score.

In one embodiment, in order to determine quality of sleep, electrodes may be placed on a user's head to measure ECG in order to provide quantitative data to identify deep sleep and REM states. In some cases, a ratio of actual sleep time over intended sleep time may be used as an indicator of sleep quality. For example, if a user wanted to sleep for 20 minutes but actually only slept for 12 minutes the sleep session would not be characterized as being good. On the other hand, if a user actually slept for 18 minutes having intended to sleep for 20 minutes then such a sleep session would be considered good.

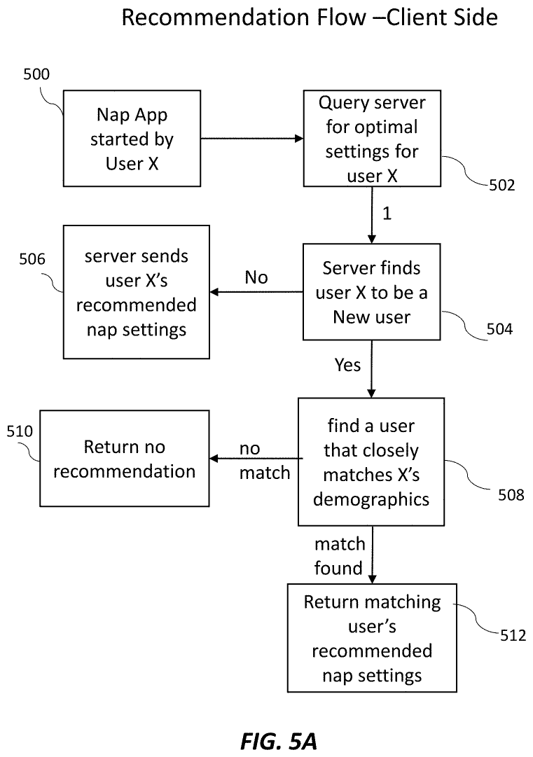

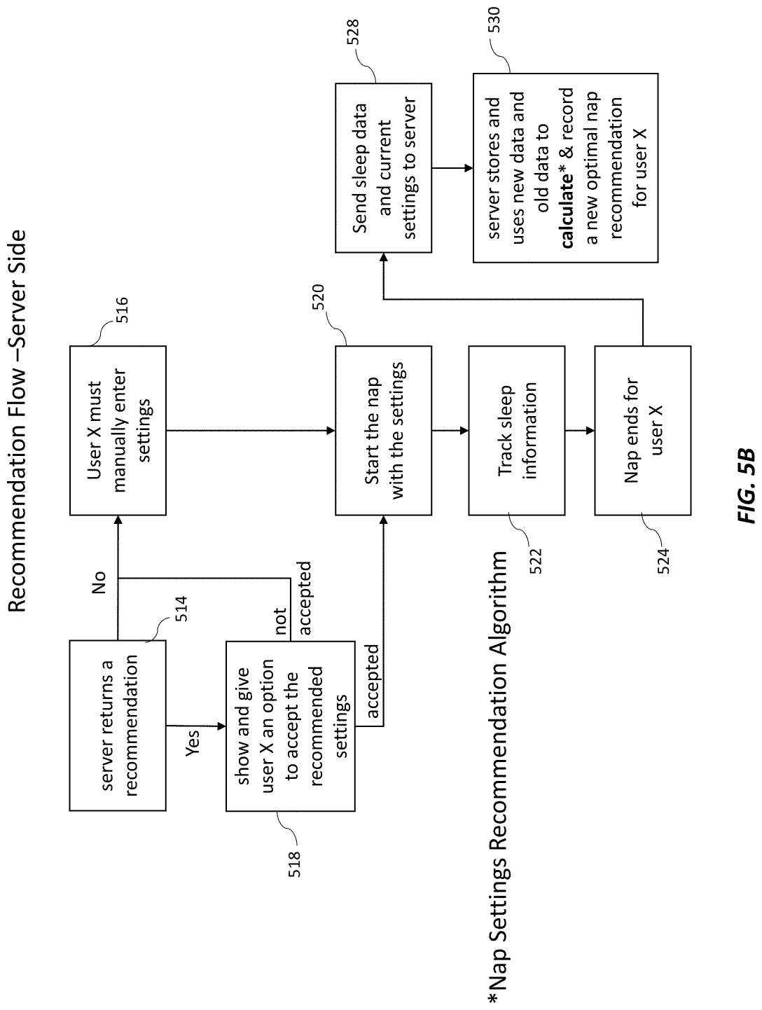

Referring now to FIG. 5A. of the drawings, there is shown a flow chart of operations performed in accordance with one embodiment of the invention in order to obtain sleep recommended settings. As will be seen, and block 500 the client software 108 (nap app) is started by a user. At block 502 the software 108 sends a query to the server device 104. The query is for optimal sleep session settings for the user. At block 504, the server (more particularly, the server application 110) initiates a lookup for a sleep profile for the user. If it is determined that a sleep profile exists, and hence that the user is not a new user, then control branches to block 506. At the block 506, the server sends recommended sleep session settings for the user. Said sleep session settings are extracted from the user's sleep profile. From the block 504, control passes to the block 508, if it is determined that the user is a new user having no stored sleep profile. In one embodiment, at the block 508, a matching process is undertaken in order to find another matching user in the system whose sleep profile could be used to generate sleep session recommended settings for the new user. In one embodiment, demographic information may be used in order to identify the matching user. For example, the matching user may be a user who shares demographic information in common with the new user. Such demographic information, in one embodiment may include (gender, race, age, annual income, number of children, marital status, hobbies, etc.) If the search at block 508 returns no matching user, then control passes to block 510 where the server device 104 returns a response to the virtual reality device 102 indicating a null recommendation, otherwise control passes to block 512 where the matching user's sleep session recommended settings is returned. Exemplary sleep recommended settings generated by the server application 110 are provided below:
a) Start sleep: between 1 pm and 1.30 pm
b) Position: 45 degrees
c) Scene (environment): Beach
d) Environment volume: 4/10
e) Background music: Galaxy Whispers
f) Background music volume: 6/10
g) Theta waves: on

Referring now to. FIG. 5B of the drawings, the client software 108 determines at block 514 whether the server device 104 has returned recommended settings for a sleep session in response to the query. If no sleep session recommended settings have been returned, then at block 516, the user may be prompted to enter sleep settings manually. Otherwise, the block 518 is executed where the received sleep recommended settings from the server device 104 are shown to the user, and the user is given an option to override or accept sleep recommended settings. Control then passes to the block 520, where a nap session is started with the final session settings. At block 522, information pertaining to the sleep session is tracked or logged. Said information may include the duration of the session, breaks in sleep, and posture (determined by gyroscope information relating to the user's neck position). A user's movement may also be tracked. In one embodiment, a user's movement may be used as a proxy to determine sleep and awake times. This translation from movement to “sleep and wake times” is based on actigraphy which is a method used to study the evaluation of sleep-wake patterns and circadian rhythms by assessing the movement recorded on an accelerometer.

Upon a determination that the sleep session has ended at block 524, the block 528 executes wherein the sleep data and the current sleep session settings are transmitted to the server device 104. In response, at block 530, the server stores the data and uses the data to generate new sleep recommended settings for the user based on computations by the sleep optimization component 304.

Advantageously, the techniques, systems, and components described herein allow users to take a nap at any time of day with optimal sleep settings so that each sleep session is efficient and provides a good quality of sleep.

In one embodiment, the sleep optimization module 304 may be configured to place users into cohorts based on metrics of similarity such as demographic information, geographic information etc. in order to identify sleep patterns across users in a particular cohort thereby to obtain sleep recommended settings for users in the cohort.

In accordance with one embodiment of the invention, users are put into groups and if a target user for whom sleep recommended settings are to be generated matches a particular group (for example, men, age 20-25), the recommendations for that group are used to determine the sleep recommended settings for the target user. Suppose there are 50 people in that group, then the 50 settings recommendations that exist for that group may be combined to give one recommendation for the target user. This may be achieved by taking averages (for things like volume and position) and MAX occurrences (for things like scene selection or music selection, theta on/off, sleep start period).

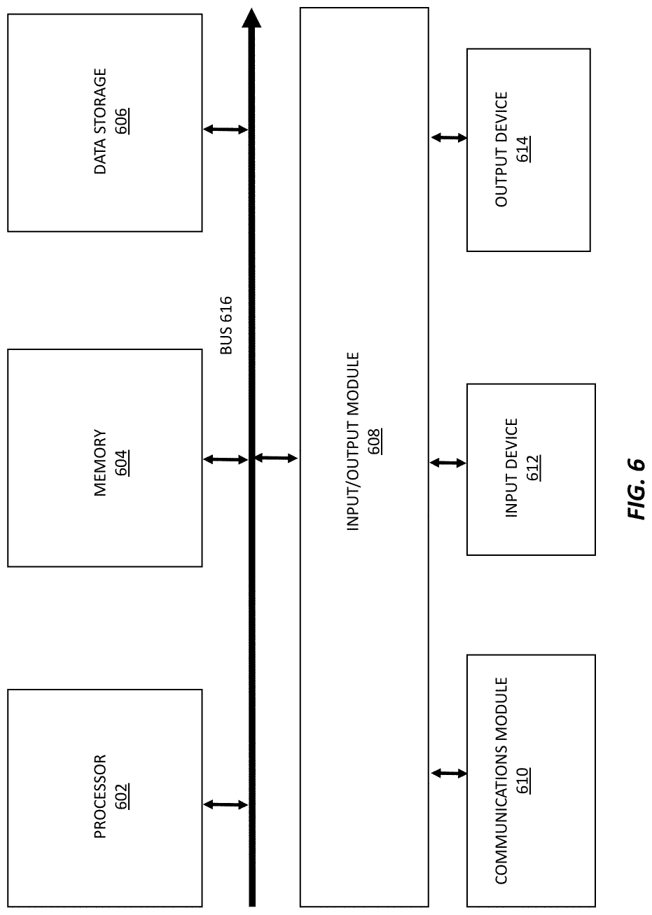

FIG. 6 is a block diagram illustrating exemplary hardware for executing some of the techniques disclosed herein, in accordance with one embodiment of the invention. In certain aspects, the computer system 600 may be implemented using hardware or a combination of software and hardware, either in a dedicated server or integrated into another entity or distributed across multiple entities.

Computer system 600 (e.g., client or server) includes a bus 608 or other communication mechanism for communicating information, and a processor 602 coupled with bus 616 for processing information. According to one aspect, the computer system 600 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques. By way of example, the computer system 600 may be implemented with one or more processors 602. Processor 602 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

Computer system 600 can include, in addition to hardware, code that creates an execution environment for the computer program in question, e.g., code that constitutes processor firmware, a protocol stack, a database management system, an operating system, or a combination of one or more of them stored in an included memory, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 616 for storing information and instructions to be executed by processor 602. The processor 602 and the memory 604 can be supplemented by, or incorporated in, special purpose logic circuitry. Expansion memory may also be provided and connected to computer system 600 through input/output module 608, which may include, for example, a SIMM (Single in Line Memory Module) card interface. Such expansion memory may provide extra storage space for computer system 600 or may also store applications or other information for computer system 600. Specifically, expansion memory may include instructions to carry out or supplement the processes described above and may include secure information also. Thus, for example, expansion memory may be provided as a security module for computer system 600 and may be programmed with instructions that permit secure use of computer system 600. In addition, secure applications may be provided via the SIMM cards, along with additional information, such as placing identifying information on the SIMM card in a non-hackable manner.

The instructions may be stored in the memory 604 and implemented in one or more computer program products, i.e., one or more modules of computer program instructions encoded on a computer readable medium for execution by, or to control the operation of, the computer system 600, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, embeddable languages, and xml-based languages. Memory 604 may also be used for storing temporary variable or other intermediate information during execution of instructions to be executed by processor 602.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 600 further includes a data storage device 606 such as a magnetic disk or optical disk, coupled to bus 616 for storing information and instructions. Computer system 600 may be coupled via input/output module 608 to various devices. The input/output module 608 can be any input/output module. Example input/output modules 608 include data ports such as USB ports. In addition, input/output module 608 may be provided in communication with processor 602, so as to enable near area communication of computer system 600 with other devices. The input/output module 608 may provide, for example, for wired communication in some implementations, or for wireless communication in other implementations, and multiple interfaces may also be used. The input/output module 608 is configured to connect to a communications module 610. Example communications modules 610 include networking interface cards, such as Ethernet cards and modems.

The components of the system can be interconnected by any form or medium of digital data communication, e.g., a communication network. The communication network can include, for example, any one or more of a PAN, a LAN, a CAN, a MAN, a WAN, a BBN, the Internet, and the like. Further, the communication network can include, but is not limited to, for example, any one or more of the following network topologies, including a bus network, a star network, a ring network, a mesh network, a star-bus network, tree or hierarchical network, or the like.

For example, in certain aspects, communications module 610 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS) messaging, CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 610 sends and receives electrical, electromagnetic or optical signals that carry digital data streams representing various types of information. The network link typically provides data communication through one or more networks to other data devices. For example, the network link of the communications module 610 may provide a connection through local network to a host computer or to data equipment operated by an Internet Service Provider (ISP). The ISP in turn provides data communication services through the world-wide packet data communication network now commonly referred to as the Internet. The local network and Internet both use electrical, electromagnetic or optical signals that carry digital data streams. The signals through the various networks and the signals on the network link and through communications module 610, which carry the digital data to and from computer system 600, are example forms of transmission media.

Computer system 600 can send messages and receive data, including program code, through the network(s), the network link and communications module 610. In the Internet example, a server might transmit a requested code for an application program through Internet, the ISP, the local network and communications module 610. The received code may be executed by processor 602 as it is received, and/or stored in data storage 606 for later execution.

In certain aspects, the input/output module 608 is configured to connect to a plurality of devices, such as an input device 612 and/or an output device 614. Example input devices 612 include a stylus, a finger, a keyboard and a pointing device, e.g., a mouse or a trackball, by which a user can provide input to the computer system 600. Other kinds of input devices 612 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 614 include display devices, such as a LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 614 may comprise appropriate circuitry for driving the output device 614 to present graphical and other information to a user.

According to one aspect of the present disclosure, the user systems and the server shown in FIG. 1 can be implemented using a computer system 600 in response to processor 602 executing one or more sequences of one or more instructions contained in memory 604. Such instructions may be read into memory 604 from another machine-readable medium, such as data storage device 606. Execution of the sequences of instructions contained in main memory 604 causes processor 602 to perform the process steps described herein. One or more processors in a multi-processing arrangement may also be employed to execute the sequences of instructions contained in memory 604. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components.

Computing system 600 can include clients and servers. A client and server are generally remote from each other and typically interact through a communication network. The relationship of client and server arises by virtue of computer programs running on the respective computers and having a client-server relationship to each other. Computer system 600 can be, for example, and without limitation, a desktop computer, laptop computer, or tablet computer. Computer system 600 can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions or data to processor 602 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 606. Volatile media include dynamic memory, such as memory 604. Transmission media include coaxial cables, copper wire, and fiber optics, including the wires that comprise bus 616. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used in this specification of this application, the terms “computer-readable storage medium” and “computer-readable media” are entirely restricted to tangible, physical objects that store information in a form that is readable by a computer. These terms exclude any wireless signals, wired download signals, and any other ephemeral signals. Storage media is distinct from but may be used in conjunction with transmission media. Transmission media participates in transferring information between storage media. For example, transmission media includes coaxial cables, copper wire and fiber optics, including the wires that comprise bus 616. Transmission media can also take the form of acoustic or light waves, such as those generated during radio-wave and infra-red data communications. Furthermore, as used in this specification of this application, the terms “computer”, “server”, “processor”, and “memory” all refer to electronic or other technological devices. These terms exclude people or groups of people. For the purposes of the specification, the terms display or displaying means displaying on an electronic device.

To illustrate the interchangeability of hardware and software, items such as the various illustrative blocks, modules, components, methods, operations, instructions, and algorithms have been described generally in terms of their functionality. Whether such functionality is implemented as hardware, software or a combination of hardware and software depends upon the particular application and design constraints imposed on the overall system. Skilled artisans may implement the described functionality in varying ways for each particular application.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. Phrases such as an aspect, the aspect, another aspect, some aspects, one or more aspects, an implementation, the implementation, another implementation, some implementations, one or more implementations, an embodiment, the embodiment, another embodiment, some embodiments, one or more embodiments, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

A reference to an element in the singular is not intended to mean “one and only one” unless specifically stated, but rather “one or more.” The term “some” refers to one or more. Underlined and/or italicized headings and subheadings are used for convenience only, do not limit the subject technology, and are not referred to in connection with the interpretation of the description of the subject technology. Relational terms such as first and second and the like may be used to distinguish one entity or action from another without necessarily requiring or implying any actual such relationship or order between such entities or actions. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description. No claim element is to be construed under the provisions of 35 U.S.C. § 112, sixth paragraph, unless the element is expressly recited using the phrase “means for” or, in the case of a method claim, the element is recited using the phrase “step for.”

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The title, background, brief description of the drawings, abstract, and drawings are hereby incorporated into the disclosure and are provided as illustrative examples of the disclosure, not as restrictive descriptions. It is submitted with the understanding that they will not be used to limit the scope or meaning of the claims. In addition, in the detailed description, it can be seen that the description provides illustrative examples and the various features are grouped together in various implementations for the purpose of streamlining the disclosure. The method of disclosure is not to be interpreted as reflecting an intention that the claimed subject matter requires more features than are expressly recited in each claim. Rather, as the claims reflect, inventive subject matter lies in less than all features of a single disclosed configuration or operation. The claims are hereby incorporated into the detailed description, with each claim standing on its own as a separately claimed subject matter.

The claims are not intended to be limited to the aspects described herein but are to be accorded the full scope consistent with the language claims and to encompass all legal equivalents. Notwithstanding, none of the claims are intended to embrace subject matter that fails to satisfy the requirements of the applicable patent law, nor should they be interpreted in such a way.

1. A method for facilitating a sleep session, the method comprising:
provisioning a virtual reality device with at least one sleep program configured to provide an immersive experience to facilitate a sleep session for a user;
determining recommended sleep settings for the sleep program;
configuring the sleep program with at least some of the recommended sleep settings;
running the sleep program; and
tracking sleep information for the user associated with the sleep session.

2. The method of claim 1, wherein determining the recommended sleep settings for the sleep program comprises obtaining said recommended sleep settings based on a query to a server application.

3. The method of claim 2, further comprising performing analysis by the server application to derive the recommended sleep settings.

4. The method of claim 3, wherein the analysis comprises learning the recommended sleep settings for a user based on historical sleep quality data, and sleep session setting data.

5. The method of claim 4, wherein said learning is based on a neural network configured to implement at least one of an Energy Based Model (EBM) and Restricted Boltzman Machine (RBM).

6. The method of claim 1, wherein the recommended sleep settings comprise settings selected from the group consisting of sleep time, sleep position, sleep environment (corresponding to a virtual reality scene), environment volume, background music, background music volume, and theta waves (on/off).

7. The method of claim 4, wherein the sleep quality data comprises a ratio of intended sleep time over actual sleep time for each sleep session.

8. The method of claim 1, wherein configuring the sleep program comprises displaying the recommended sleep settings to the user; and allowing the user to accept or override said recommended sleep settings prior to the sleep session.

9. The method of claim 4, wherein learning the recommended sleep settings comprises generating recommended sleep settings for the user based on sleep settings associated with a group into which the user is classified based on a metric of similarity.

10. The method of claim 9, wherein the metric of similarity comprises demographic information.

11. A system for facilitating a sleep session, the system comprising:
a virtual reality device provisioned with at least one sleep program configured to provide an immersive experience to facilitate a sleep session for a user;
a server device communicatively coupled to the virtual reality device and configured to provide recommended sleep settings for the sleep program.

12. The system of claim 11, further comprising a plurality of virtual reality devices each associated with a user, wherein each virtual reality device tracks sleep session data for each sleep session and transmits same to the server device.

13. The system of claim 12, wherein the server device is configured to learn recommended sleep settings for each user based on the sleep session data.

14. The system of claim 12, wherein the server device is configured to learn the recommended sleep settings based on a neural network.

15. The system of claim 12, wherein the neural network implements a probabilistic model to learn the sleep settings for each user corresponding to the best quality of sleep for that user.

16. The system of claim 15, wherein the best quality of sleep for a user is learned based on historical sleep session data.

17. The system of claim 16, wherein quality of sleep for each user is inferred based on a ratio of intended sleep time over actual sleep time for each sleep session.

18. The system of claim 14, wherein the server device is further configured to classify each user into a cohort based on a metric of similarity.

19. The system of claim 18, wherein the metric of similarity includes demographic information.

20. The system of claim 18, wherein for selected instances sleep session recommended settings for a user are generated based on sleep session settings for the cohort which the user is classified
